(19) United States
(12) Patent Application Publication
(10) Pub. No.: US 2020/0233109 A1
(43) Pub. Date: Jul. 23, 2020

(54) LIQUID COUPLED ULTRASONIC TRANSDUCER ARRAY FOR MEASUREMENT OF ROCK ELASTIC PROPERTIES

(71) Applicant: BOARD OF REGENTS, THE UNIVERSITY OF TEXAS SYSTEM, AUSTIN, TX (US)

(72) Inventors: Carlos TORRES-VERDÍN, Austin, TX (US); Adam James MCMULLEN, The Hills, TX (US)

(21) Appl. No.: 16/639,175

(22) PCT Filed: Aug. 13, 2018
(86) PCT No.: PCT/US2018/046467

§ 371 Date: Feb. 14, 2020

Related U.S. Application Data
(60) Provisional application No. 62/545,156, filed on Aug. 14, 2017.

(51) Int. Cl. G01V 1/30; G01V 1/38; G01N 33/24

(52) U.S. Cl. CPC G01V 1/303; G01V 1/306; G01V 1/38; G01V 2210/6161; G01V 2210/6222; G01V 2210/584; G01N 33/24

(57) ABSTRACT

Disclosed and described herein are systems and methods used to analyze ultrasonic waves and nondestructively infer acoustic wave velocities and dynamic elastic properties of materials. Disclosed methods employ the selective rotation of ultrasonic transducers immersed in a liquid (water) adjacent to the sample under study.

### CROSS REFERENCE TO RELATED APPLICATION

This application claims priority to and benefit of U.S. provisional patent application Ser. No. 62/545,156 filed Aug. 14, 2017, which is fully incorporated by reference and made a part hereof.

### BACKGROUND

Conventional ultrasonic laboratory measurements are performed by attaching either P or S transducers to the flat faces of cylindrical core samples. Measurements of acoustic travel time are made via a through-transmission acoustic pulse that travels through the rock from the transmitter to receiver. Various standardizations exist to properly setup the experiments and assist selection of an ideal frequency of investigation in relation to sample size [1]. The main components required are a pulse or signal generator unit, the transducers, and an oscilloscope to read and save the signals for processing.

Today, oil/gas markets are more volatile than ever, and the traditional development process hardly meets rapidly-changing industry needs.

### SUMMARY

Various aspects of the disclosure provide new techniques to analyze rock P and S-wave velocities. For instance, consider seismic refraction surveys and multiple receiver acoustic logging techniques. Both methods utilize the physics of refraction and mode conversion, which occurs at the interface between elastic media [2]. In both systems, interactions are governed by Snell's Law, which explains how the source P-wave will refract in the formation at specific angles as a function of the layer velocities and incident angle. To capitalize on the fundamentals of borehole acoustics and refraction, this work will present the development of a transducer array system that treats the sample as an inverted borehole, in which the sample is immersed in water and the transducers are external to the sample.

Pressure-coring technology, such as the HYACINTH system, allows for the study of core samples at the surface while retaining in-situ pressures and temperatures [3]. At the surface, the system measures P-wave velocity across the diameter of the cylindrical core sample using complex rolling transducers [4]. Currently, minimal standardizations exist for measuring core P-wave velocity in this fashion. As such, this type of system will also be studied to better understand the benefits and limitations. Various dry and water saturated cores are tested using both experimental systems, with results corroborated using Gassmann's equations [5].

The disclosed embodiments allow for acoustic velocity analysis of a full-core without contacting or damaging the sample. It may be used on cores within a core liner or in a pressure vessel to better preserve fluid saturations. Because the technology can analyze the entire core, it may allow easier comparison between wireline or LWD acoustic logging and core data. This system could supplement routine core analysis acoustic data and may provide service companies with a simple, non-destructive core testing protocol to characterize the P and S-wave velocities of a full-core sample with ease. It may also prove useful in highlighting local acoustic heterogeneity which conventional wireline logs or biased core plug sampling may overlook, especially in thinly bedded formations.

Typical ultrasonic analysis of rocks requires direct transducer sample contact at the flat ends of a cylindrical core sample, often under high force with a coupling gel to ensure proper bonding. Th disclosed embodiments of a system are advantageous in that the sample under investigation does not have to be rigidly connected to the transducers, which greatly simplifies testing and reduces user error.

The disclosed methods present a possibility of measuring the entire length of the core in the lab without having to cut the core into pieces, which may be used to generate a core log of acoustic velocity or slowness. Additionally, it would be feasible to rotate the sample along its axis and generate a more detailed circumferential acoustic velocity profile at each core depth to provide insight into the sample heterogeneity; compared to typical ultrasonic measurements, this proposed form of analysis may provide greater detail than the conventional entire core average.

In cases where a core sample is environmentally isolated to preserve in-situ properties, such as in pressure coring applications, the core cannot be removed and must be examined often through core liners or within pressure vessels. The disclosed system embodiments can be directly integrated into current pressure-coring technologies with ease, perhaps with multiple sensors integrated around the entire core circumference. Present pressure coring technologies use transducers that measure the P or S velocity directly across the core sample diameter, which rely on difficult to achieve sample transducer contact through a core liner.

These and other features will be more clearly understood from the following detailed description taken in conjunction with the accompanying drawings and claims.

### BRIEF DESCRIPTION OF THE DRAWINGS

FIG. 1A is an image that illustrates an embodiment of a system that hold one or more (in this example, three) ultrasonic compressional wave transducers adjacent to a sample under investigation.

FIG. 1B is an image that shows the parts of FIG. 1A assembled.

FIGS. 2A-2C illustrate details of the nut, tapered machine screw, and washer assembly that is used to hold the transducer holder at a certain height.

FIG. 3 shows a schematic of transducer with P (red), S (green), and direct water (blue) wave ray traces.

FIGS. 4A-4D illustrate error in velocity estimation vs. arrival time estimation for all samples tested at both transducers for P and S-waves.

FIG. 5A shows the individual system components, with the transducer mounts and brackets identical to those used in the first embodiment.

FIG. 5B illustrates a sample experiment setup with a flat sample of Berea sandstone.

FIG. 6 illustrates the reflection coefficient, Rₚ, as a function of incidence angle for various samples with a comparison to Aki-Richard's approximation. Blue: aluminum; Red: Berea sandstone; Green: Texas Cream limestone, where (+) are 4 in OD core samples, and (×) 2×2×3 in rectangular samples.

FIG. 7A illustrates 2D forward model schematic of prototype No. 1 and FIG. 7B illustrates the same for prototypes No. 2 and 3.

FIGS. 8A-8D are graphs illustrating that the error in arrival time estimation is consistently under 4%, and the error in velocity estimation is under 6%.

For a more complete understanding of the present disclosure, reference is now made to the following brief description, taken in connection with the accompanying drawings and detailed description, wherein like reference numerals represent like parts.

### DETAILED DESCRIPTION

It should be understood at the outset that although illustrative implementations of one or more embodiments are illustrated below, the disclosed tools and methods may be implemented using any number of techniques, whether currently known or in existence. The disclosure should in no way be limited to the illustrative implementations, drawings, and techniques illustrated below, but may be modified within the scope of the appended claims along with their full scope of equivalents. Use of the phrase “and/or” indicates that anyone or any combination of a list of options can be used. For example, “A, B, and/or C” means “A”, or “B”, or “C”, or “A and B”, or “A and C”, or “A and B and C”.

Disclosed and described herein are systems and methods used to analyze ultrasonic waves and nondestructively infer acoustic wave velocities and dynamic elastic properties of materials. Both methods employ the selective rotation of ultrasonic transducers immersed in a liquid (water) adjacent to the sample under study.

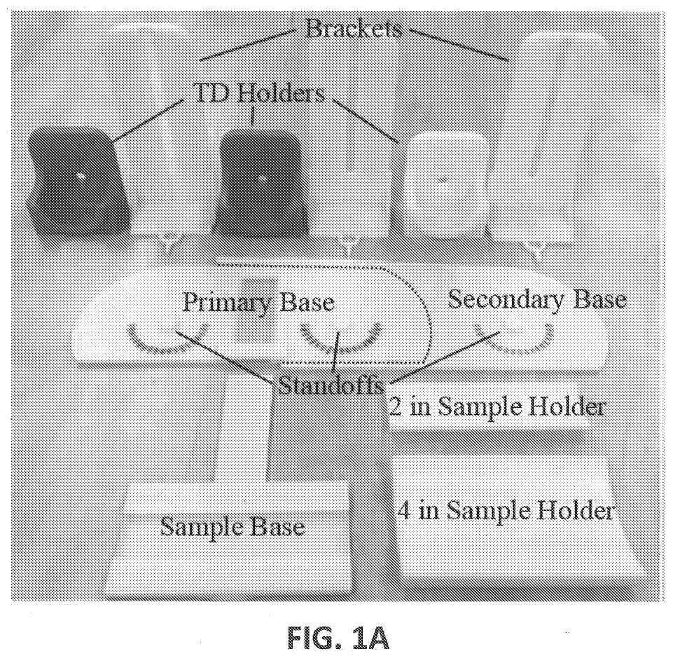

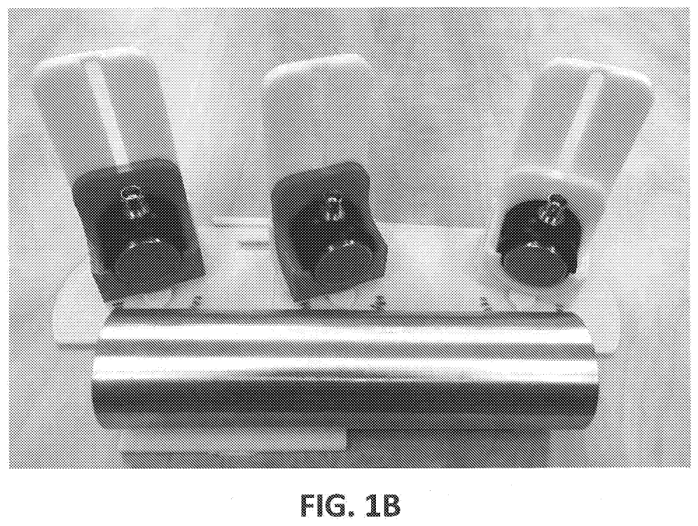

FIG. 1A is an image that illustrates an embodiment of a system that hold one or more (in this example, three) ultrasonic compressional wave transducers adjacent to a sample under investigation. The transducer height and angle of incidence are adjustable. FIG. 1A shows the individual parts of one embodiment of the system, including three transducer (TD) holders, three transducer mounting brackets, a primary and secondary base with circular standoffs (a rotatable joint), and a sample base which is compatible with either a 2 in or 4 in sample holder. Note the angle markings adjacent to the standoff, which are used to set the transducer angle during experimentation.

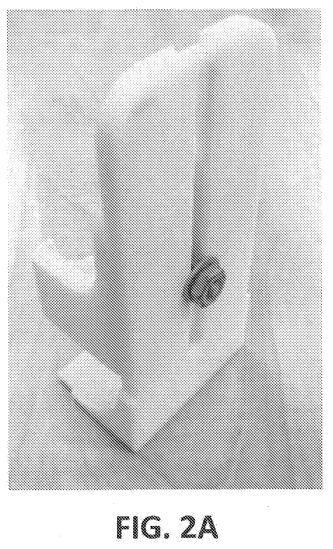

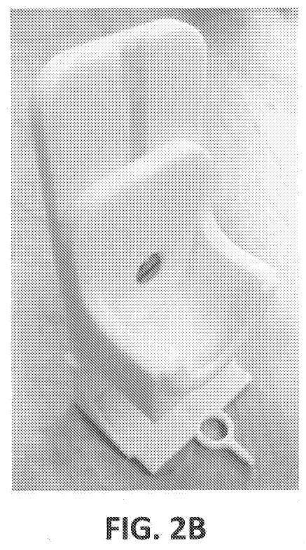

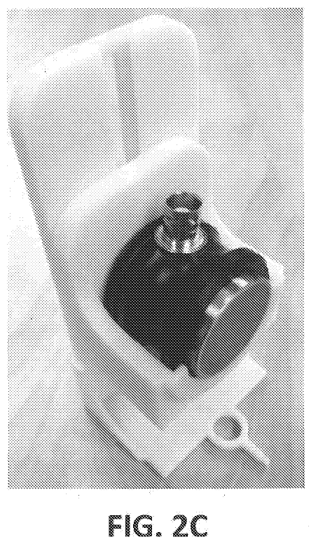

FIG. 1B is an image that shows the parts of FIG. 1A assembled. FIG. 1B illustrates three transducers rotated at 20° at the default height (transducer center height of 1 in, i.e., center of 2 in outer diameter (OD) sample) and a cylindrical 2 in OD aluminum sample. In FIG. 2B, the system is assembled using a 2 in sample holder with a 2 in outer diameter (OD) cylindrical aluminum sample. The prototype makes use of tapered sliding connections that allow linear translation to optimize testing flexibility. The directions of translation are as follows: 1) the transducer holders may travel up to 3.5 in in height (z direction), with the default center height aligned with the center of a 2 in OD sample (1 in); 2) the sample base can extend away from the primary base up to 4 in (y direction) to accommodate larger samples; and 3) the secondary base can extend up to 4 in (x direction) away from the primary base.

FIGS. 2A-2C illustrate details of the nut, tapered machine screw, and washer assembly that is used to hold the transducer holder at a certain height. FIG. 2A shows the rear of transducer bracket/mount assembly showing bolt and washer, FIG. 2B shows the front assembly showing the flush tapered screw and the standoff mounting hole with angle selector, and FIG. 2C shows the transducer bracket/mount assembly with a transducer installed. It is desired to align the transducers to samples of varying size.

The above-described system is used to investigate both the compressional (P-wave) and shear (S-wave) velocity of elastic media. For the disclosed application, the focus is on rock samples because acoustic velocities are associated with rock elastic properties, density, and capacity to store hydrocarbons (porosity).

The mode of operation comprises submerging the entire assembly, including sample, in a liquid (e.g., water). The liquid acts as a couplant between the transducers and the sample, which effectively transmits ultrasonic energy. Transducer 1 (FIG. 1B) acts as the emitter, while transducers 2 and 3 act as receivers. Using a waveform generator, a square wave pulse corresponding to the resonant frequency of the transducers (500 kHz) is sent to the emitter. An oscilloscope records the resulting waveforms received at transducers 2 and 3, which are time referenced to the pulse sent to transducer 1. In this way, the system measures the time of flight of the refracted P-wave and S-wave. However, to do so effectively requires an understanding of Snell's Law and careful consideration of the geometry. Namely, Snell's Law governs the physics of refraction that occur at the water-sample interface. This allows for calculation of the critical angle at which the transducer must be rotated to induce a critically refracted wave with the highest energy. This is why the transducer rotation is important, as it is used to ‘dial in’ the appropriate critical angle to induce this highest amplitude wave and cleanest response. This allows further validation of the P and S-wave velocities of the sample. The system is designed such that transducer rotation occurs exactly at the transducer center because this better constrains the system geometry as the transducer faces are rotated.

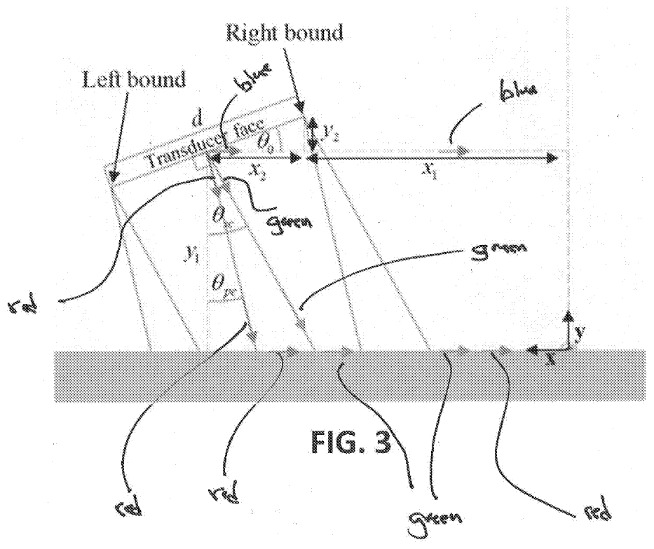

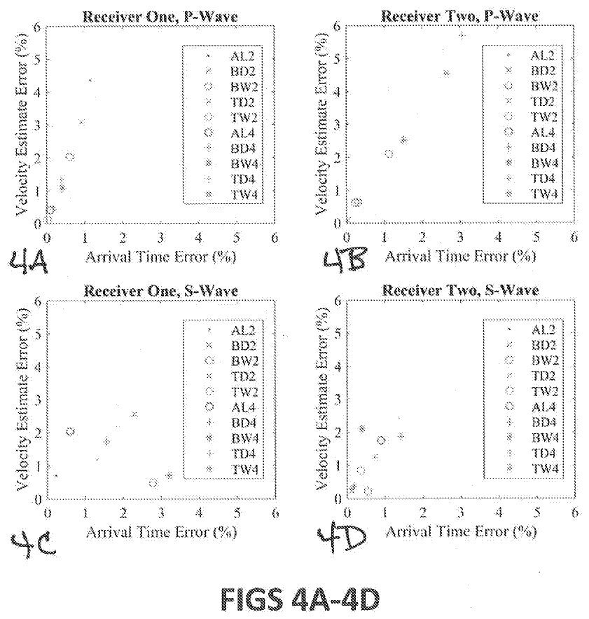

Prior to testing, the geometry of the system is measured such that the time of flight can be appropriately modeled in MATLAB in 2D, as shown in FIG. 3. FIG. 3 shows a schematic of transducer with P (red), S (green), and direct water (blue) wave ray traces. The only dimensions required for modelling are the sample to center transducer spacing (y₁) and half the inter-transducer spacing (x₁+x₂). The total receiver spacing (2(x₁+x₂)) is fixed at 100 mm (transducer 2 to 3 in FIG. 1B), but the emitter-receiver spacing (transducer 1 to 2) may vary from 100 to 200 mm, depending on whether or not the user would like to avoid the presence of reflected modes arriving at transducer 2 at larger angles of investigation. With these dimensions known, the system may be forward modelled in the time domain based on assuming the respective speeds of sound in water and the sample for both P and S-waves. Laboratory data are collected at various transducer rotation angles, θ₀, and the forward model predictions of arrival time are superimposed on the waveforms. The data show excellent agreement (<5% error) and suggest that this method can be used to measure rock P and S acoustic velocities simultaneously. See FIGS. 4A-4D for a summary of the error in arrival time and velocity estimation. FIGS. 4A-4D illustrate error in velocity estimation vs. arrival time estimation for all samples tested at both transducers for P and S-waves.

While the forward model is useful for understanding the data, this system and method has an added bonus of reducing the need for the detailed forward model. By comparing the waveforms arriving at transducers 2 and 3, and with a knowledge of the inter-receiver spacing, the P and S-wave velocities may be estimated by simply dividing the spacing (distance) over the difference in arrival time.

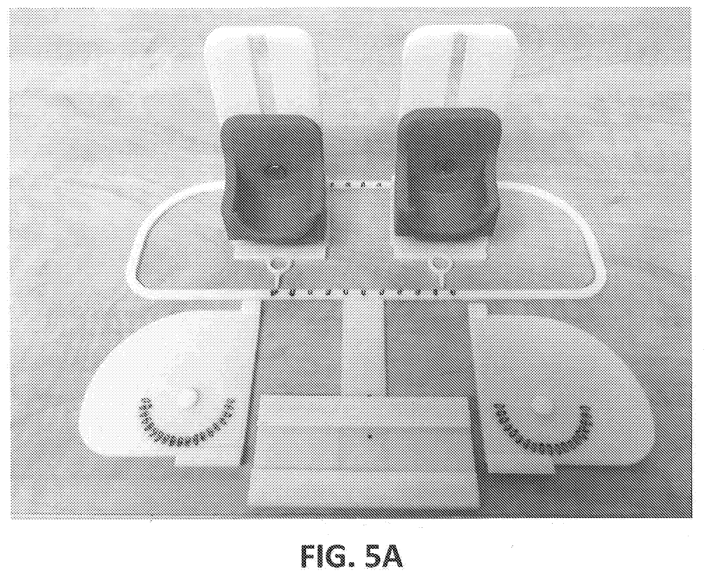

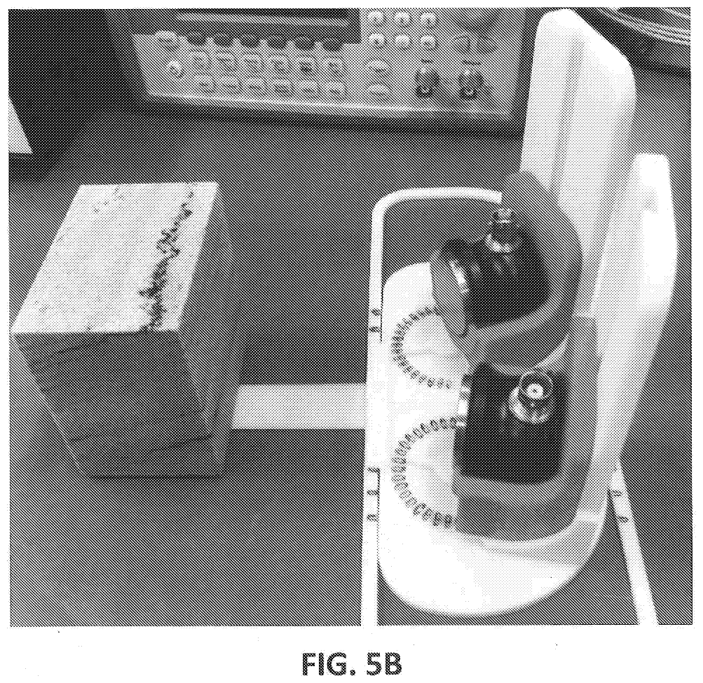

A second disclosed embodiment uses a similar method of operation. However, the purpose of this system is to probe for directly reflected waves. FIG. 5A shows the individual system components, with the transducer mounts and brackets identical to those used in the first embodiment. The slide base with separation markings (10 mm spacing) allows for modification of the transducer separation or offset. The transducer separation may vary from 60 mm to 160 mm, and the transducer angle can similarly be modified. FIG. 5B illustrates a sample experiment setup with a flat sample of Berea sandstone.

This embodiment of a method relies on analysis of the amplitude of the P-wave reflections, akin to seismic analysis. This allows indirect analysis of the sample P and S-wave velocities via the Zoeppritz equations. The amplitude of reflection as a function of angle is the only pertinent variable, which greatly reduces analytical complexity. The method of analysis is as follows: 1) With knowledge of the transducer separation and sample to transducer separation, calculate the angles of incidence associated with the transducer spacing increments. 2) Dial in the appropriate angle at each transducer separation (offset) and measure the P-wave reflection. 3) After accounting for attenuation due to the water and with knowledge of the initial amplitude of the wavelet, estimate the reflection coefficient. 4) Compare the amplitude results as a function of angle to theoretical models, such as the Aki-Richards' approximation.

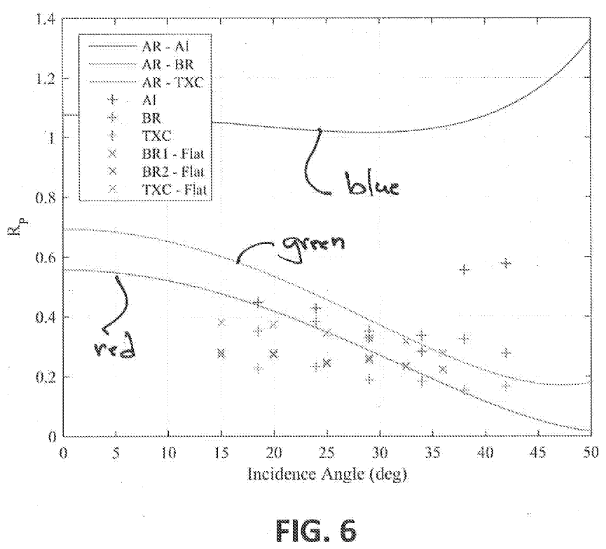

In this way, the method effectively estimates the sample P and S-wave velocities. Results for this are preliminarily shown in FIG. 6. The solid lines are the Aki-Richard's approximation models, and the points show experimental data. Tests on 4 in OD aluminium show a trend similar to the theory, but the cylindrical rock samples do not readily agree. Flat rock samples (crosses) show a much more improved response; note the predicted magnitude of reflection difference between Berea sandstone and Texas Cream limestone agreement with the data. FIG. 6 illustrates the reflection coefficient, Rₚ, as a function of incidence angle for various samples with a comparison to Aki-Richard's approximation. Blue: aluminum; Red: Berea sandstone; Green: Texas Cream limestone, where (+) are 4 in OD core samples, and (×) 2×2×3 in rectangular samples.

Experimental Setup

A system of 3D printed parts hold the transducers in place and ensure their alignment when adjacent the sample. Prototype No. 1 allows the user to adjust the transducer center height and take P-wave velocity measurements across the sample diameter. Since the smallest diameter samples tested are 2 in, the default resting center height of the transducers is designed to correspond to 1 in, i.e., the sample mid-plane for a 2 in OD sample. This greatly simplifies operation, and adjustments in height are readily made via caliper. The system is easy to implement or assemble submerged in water, as is the case for all experiments. Water is used to properly couple the transducers to the outer diameter of the sample because air does not readily transmit ultrasonic energy through the sample. Prototype No. 1 is used to evaluate modern pressure-core P-wave arrival time estimation techniques. Namely, the variation in sample velocity estimation as a function of transducer height is evaluated. This is important because currently there are minimal standardizations that exist for examining acoustic velocity in this manner. This prototype will also evaluate the effectiveness of the liquid coupling.

Prototype No. 2 allows transducers to rotate adjacent to the sample and probe for refracted and converted waves. One major drawback is that the rotation joint causes the transducer center point to follow an arc during rotation. Prototype No. 3 addresses this issue, and also introduces a second receiver as dual array system. Transducer rotation occurs precisely at the transducer center point, which greatly simplifies forward modelling and testing. Prototype No. 3 can test angles up to 65° using both receivers.

All experiments were performed on the following materials: Aluminum 2024, Berea sandstone, and Texas Cream limestone. The samples of each material are approximately 2 and 4 in OD by 8 in length. The aluminum functions as an ideal homogenous, isotropic media that is used as a control. The sample length is important for testing with prototype three, but inconsequential for testing with prototype one. Namely, the transducer size and resulting size of prototype No. 3 dictate the use of relatively long samples.

The rock samples were tested dry and fully water saturated. The samples were dried in an oven for 48 hours prior to any testing or water saturation procedure. To create fully water saturated cores, the 1998 API Recommended Practices for Core Analysis liquid saturation guidelines were followed [6].

Prototype No. 1 estimates P-velocity similarly to existing pressure coring systems. To evaluate the effectiveness of this technique, experiments will change the transducer center height with respect to the sample. This height, differential will be referred to as the height offset. The experiments start with the transducer center axis aligned to the sample mid-plane, and experiments are conducted in transducer center height offset increments of 0.2 in until the transducer center axis is tangent to the top of the sample. The transducer faces are pressed against the core at each height increment, which reduces the separation distance between the transducers with increasing height.

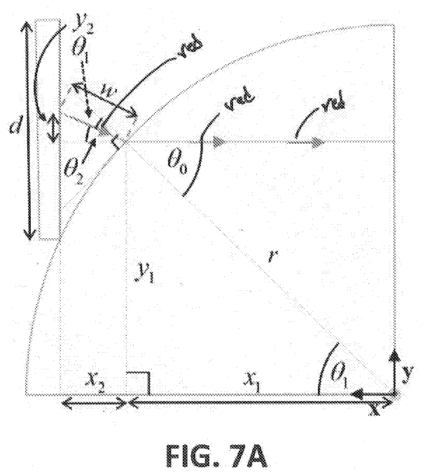

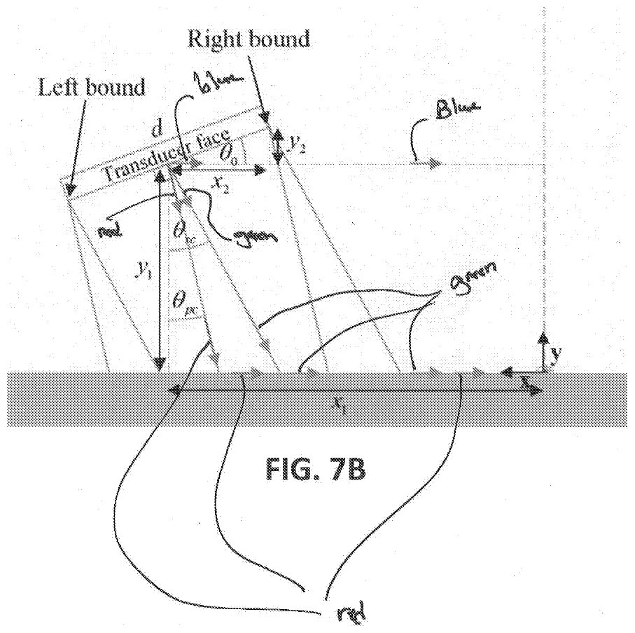

The forward model takes into account a simplified 2D system geometry and calculates the transducer separation distance as a function of height offset. The model treats the transducer face as a collection of point sources and determines the critical incident angles at which a waveform will travel parallel to the mid-plane. Model inputs are the speed of sound (P-wave velocity) in water and the sample, and the dimensions of the physical system. The model then varies the location of the transducer face, assuming that a sample contact constraint occurs at the bottom of the transducer. A point source of sound is simulated to originate across the entire transducer face of height d, and the model solves for the corresponding 60 from each point (See FIG. 7A). The model then calculates the half-distance traveled in water (w) and the half distance traveled in the sample (x₁). Based on these distances, a composite arrival time can be determined. A velocity estimate is made by dividing the total transducer separation (X_{T}=2(x₁+x₂)) by the arrival time.

Prototype No. 2 and 3 require a similar forward modeling treatment. However, detecting P and S-waves requires observation of the entire wave train, not just the first arrival. The transducer center height is fixed at the sample mid-plane for all experiments. By assuming that both transmitter and receiver(s) are rotated at the same incident angle, the system geometry is greatly simplified. FIG. 7B shows a sketch of a transducer face, with the center separated a distance x₁ and y₁ from the origin. The dark grey represents the sample, and the blue lines between the sample and transducer face correspond to the critical P and S-wave ray traces that originate from the left, center, and right of the transducer at critical angles θ_{pc} and θ_{sc}. The red and green arrows indicate the path of the critical P and S-waves from the transducer center.

At every point along the transducer face, in 2D, we assume a circular wave front propagates from the transducer. At all angles θ₀ from 0 to 90′, there will therefore be some energy transmitted at the critical angle. The transducer x and y offset (x₂ and y₂) from the transducer center is calculated at each θ₀ to determine the left and right sensor boundary points for the model. These boundary points determine the range of arrival times as a function of the distance traveled in the water and sample. Further, the model calculates the arrival time of a direct fluid mode propagating parallel to the sample (blue arrows). This is important to monitor for quality control purposes.

### CONCLUSIONS

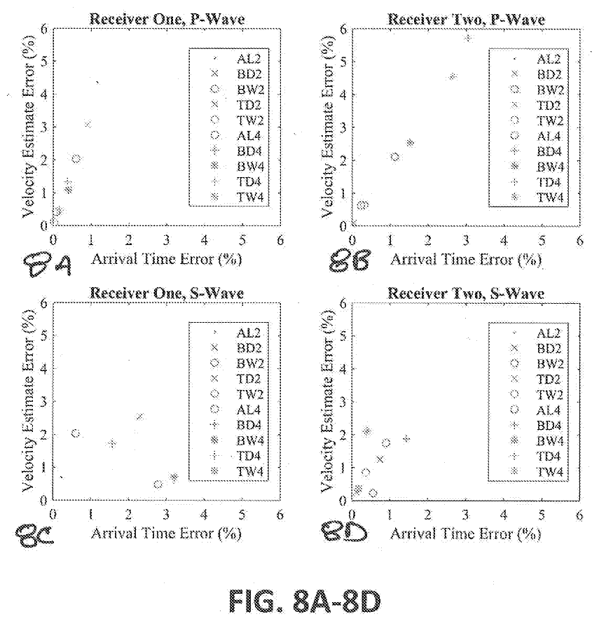

The cross-diameter P-wave measurements using prototype No. 1 suggest that there is a minimum amount of fluid required to properly couple ultrasonic energy to the sample. P-wave velocity estimates on the 4 in samples are more accurate than 2 in samples, but still underestimate the conventional acoustic velocities due to the fluid coupling. Knowledge of the thickness of fluid between the transducers and sample eliminates the underestimation. Measurements acquired on both dry and fully water saturated core samples agree with the magnitude of P-wave velocity change as predicted by Gassmann's fluid substitution theory within a 2% margin of error. Results from refracted wave tests using prototype No. 3 are ideal for all samples tested. The error in arrival time estimation is consistently under 4%, and the error in velocity estimation is under 6% (FIGS. 8A-8D). Samples with critical angles of refraction from 45 to 60° may experience interference on the first receiver due to a direct reflection. Additionally, slower samples are subject to direct fluid arrival interference for the first receiver. The use of a second receiver spaced further away from the source simplifies isolation of the relevant wave modes. The techniques are valid for slow formations if the fluid and reflected modes are properly isolated-which is the intended purpose of the second receiver. Overlain raw waveform data with forward model predictions is critical for analytical consistency.

The forward model acts as a reality check for the user to either repeat the experiment or double check the model conditions should model disagreements arise. Model utility is large ty dependent on how well the initial conditions of the experiment are measured. The forward model predictions agree with experimental results and show future potential of simultaneous P and S-wave measurement in the laboratory.

### REFERENCES

Unless otherwise noted, each of the below fully incorporated herein by reference and made a part hereof:
[1] ASTM D2845-08, Standard Test Method for Laboratory Determination of Pulse Velocities and Ultrasonic Elastic Constants of Rock (Withdrawn 2017), ASTM International.
[2] Haldorsen, J. B. U., Johnson, D. L., Plona, T., Sinha, S., Valero, H. P., and Winkler, K., 2006, Borehole Acoustic Waves, Oilfield Review 18 (1), Schlumberger.
[3] Schultheiss, P. J., Francis, T. J. G., Holland, M., Roberts, J Amann, H., Thjunjoto, Parkes, R. J., Martin, D., Rothtuss, M., Tyunder, F., and Jackson, P. D., 2006, Pressure Coring, Logging and Subsampling with the HYACINTH System, Geological Society, London, Special Publications 267 (1): 151-63.
[4] Geotek Ltd., Geotek P-Wave Sensor Technology, http://www.geotek.co.uk/products/arc
[5] Berryman, J. G. 1999, Origin of Gassmann's Equations, Geophysics 64.5:1627-629, DCI: 10.1190/1.1444667.

[6] RP 40, Recommended Practices for Core Analysis, 2nd Edition 1998, API.

In the specification and/or figures, typical embodiments have been disclosed. The present disclosure is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Those skilled in the art will also appreciate that various adaptations and modifications of the preferred and alternative embodiments described above can be configured without departing from the scope and spirit of the disclosure. Therefore, it is to be understood that, within the scope of the appended claims, the disclosure may be practiced other than as specifically described herein.

1. A method of determining a compressional (P-wave) and shear (S-wave) velocity of elastic media comprising:
placing two or more transducers in a transducer bracket/mount assembly;
align the two or more transducers with a sample adjacent to the two or more transducers;
submerging the transducer bracket/mount assembly, two or more transducers, and sample, in a liquid;
send a square-wave pulse having a frequency corresponding to a resonant frequency of one the transducers to one of the transducers that is acting as an emitter;
record resulting waveforms received at any of the two or more transducers that are not acting as the emitter, which are time referenced to the pulse sent to the transducer acting as the emitter, wherein a time of flight of a refracted P-wave and S-wave is measured using Snell's Law and consideration of an angle at which the transducer acting as an emitter must be rotated to induce a critically refracted wave with the highest energy.

2. The method of claim 1, wherein the two or more transducers comprise three transducers.

3. The method of claim 1, wherein the liquid comprises water.

4. The method of claim 1, wherein the frequency of the square-wave pulse is 500 kHz.

5. The method of claim 1, wherein the sample comprises a rock.

6. A method of determining a compressional (P-wave) and shear (S-wave) velocity of elastic media comprising:
placing two or more transducers in a transducer bracket/mount assembly;
align the two or more transducers with a sample adjacent to the two or more transducers;
submerging the entire transducer bracket/mount assembly, two or more transducers, and sample, in a liquid;
with knowledge of separation distance between the two or more transducers and sample to transducer separation distance, calculate angles of incidence associated with transducer spacing increments;
dial in an appropriate angle at each transducer separation (offset) and measure P-wave reflection;
after accounting for attenuation due to the water and with knowledge of an initial amplitude of a wavelet, estimate a reflection coefficient using Zoeppritz equations; and
compare the amplitude results as a function of angle to theoretical models, which effectively estimates the sample P and S-wave velocities.

7. The method of claim 6, wherein comparing the amplitude results as a function of angle to theoretical models comprises comparing the amplitude results as a function of angle to an Aki-Richards' approximation.

8. The method of claim 6, wherein the two or more transducers comprise two transducers.

9. The method of claim 6, wherein the liquid comprises water.

10. The method of claim 6, wherein the sample comprises a rock.

11. A system for determining a compressional (P-wave) and shear (S-wave) velocity of elastic media comprising:
two or more transducers, wherein the two or more transducers are each held in place by a transducer bracket/mount assembly that aligns the two or more transducers with a sample adjacent to the two or more transducers, wherein a transducer height and angle of incidence relative to the sample are adjustable using the transducer bracket/mount assembly;
a liquid, wherein the transducer bracket/mount assembly, two or more transducers, and the sample, are submerged in the liquid, wherein the liquid acts as a couplant between the two or more transducers and the sample to transmit ultrasonic energy;
a waveform generator, wherein the waveform generator sends a square-wave pulse having a frequency corresponding to a resonant frequency of one the transducers to the one of the transducers such that the one of the transducers is acting as an emitter; and
an oscilloscope, wherein the oscilloscope records resulting waveforms received at any of the two or more transducers that are not acting as the emitter, wherein the resulting waveforms are time referenced to the pulse sent to the transducer acting as an emitter, wherein a time of flight of a refracted P-wave and S-wave is measured using Snell's Law and consideration of an angle at which the transducer acting as an emitter must be rotated to induce a critically refracted wave with the highest energy.

12. The system of claim 11, wherein the two or more transducers comprise three transducers.

13. The system of claim 11, wherein the liquid comprises water.

14. The system of claim 11, wherein the frequency of the square-wave pulse is 500 kHz.

15. The system of claim 11, wherein the sample comprises a rock.

16. The system of claim 11 further comprising:
determining a separation distance between the two or more transducers and a sample to transducer separation distance;
calculating angles of incidence associated with transducer spacing increments;
dialing in an appropriate angle at each transducer separation (offset) and measure P-wave reflection;
estimating a reflection coefficient using Zoeppritz equations, wherein the estimation accounts for attenuation due to the liquid and with knowledge of an initial amplitude of a wavelet; and
comparing the amplitude results as a function of angle to theoretical models, which effectively estimates the sample P and S-wave velocities.

17. The system of claim 16, wherein comparing the amplitude results as a function of angle to theoretical models comprises comparing the amplitude results as a function of angle to an Aki-Richards' approximation.
